(19) United States
(12) Patent Application Publication
(10) Pub. No.: US 2017/0273898 A1
(43) Pub. Date: Sep. 28, 2017

(54) COMPOUNDED TOPICAL COMPOSITION AND METHOD

(71) Applicant: CMPD LICENSING, LLC, Conroe, TX (US)

(72) Inventor: Jay Richard Ray, II, Conroe, TX (US)

(73) Assignee: CMPD LICENSING, LLC, Conroe, TX (US)

(21) Appl. No.: 15/484,570

(22) Filed: Apr. 11, 2017

Related U.S. Application Data
(60) Provisional application No. 62/312,000, filed on Mar. 23, 2016.

(51) Int. Cl. A61K 9/00; A61K 31/522; A61K 47/46; A61K 47/10; A61K 45/06; A61K 47/34; A61K 31/167; A61K 9/06

(52) U.S. Cl. CPC A61K 9/0014; A61K 31/167; A61K 31/522; A61K 9/06; A61K 47/10; A61K 45/06; A61K 47/34; A61K 47/46

(57) ABSTRACT

A compounded topical composition may be compounded by combining a local anesthetic component with a topical base composition. The local anesthetic component may comprise lidocaine hydrochloride monohydrate and the topical base composition may comprise an Acyclovir 5% Ointment containing 5% acyclovir by weight and polyethylene glycol.

### CROSS-REFERENCE TO RELATED APPLICATIONS

The present application claims priority under 35 U.S.C. §119(e) to U.S. Provisional Patent Application No. 62/321,000 filed on Apr. 11, 2016, which is hereby incorporated by reference into this specification.

### TECHNICAL FIELD

The present application relates to compounded pharmacological therapies. In particular, the present application relates to compounded topical compositions for antiviral therapies to treat viral ailments, such as those related to herpes simplex and varicella zoster.

### BACKGROUND

Transdermal creams are employed to deliver medication to the skin of a patient. Conventional compositions intended for topical administration include EMLA cream, a eutectic mixture of lidocaine and prilocaine in an emulsified topical cream, such as disclosed by U.S. Pat. Nos. 6,299,902 and 4,562,060, which are incorporated herein by reference in their entireties. However, conventional transdermal creams may include various drawbacks, such as addressing limited medical conditions, creating adverse side effects, and/or having limited shelf lives. Additionally, conventional methods of manufacturing transdermal creams may be inefficient and/or lack precision with the amount of active ingredients, or have other drawbacks.

### SUMMARY

A compounded topical composition may include a topical base component to which one or more active components have been added and mixed. In various embodiments, active components may include an antiviral component and a local anesthetic component.

The compounded topical composition may comprise additional active components such as an anti-inflammatory component, an anticonvulsant component, a nerve depressant component, a muscle relaxant component, a NMDA (N-Methyl-D-aspartate) receptor antagonist component, an opiate or opioid agonist component, an antidepressant component, or combinations thereof.

The active components may include active ingredients in the form of powders combined with the topical base component wherein the topical base component comprises as suitable base vehicle, such as polyethylene glycol. In various embodiments, the compounded topical composition may comprise a local anesthetic component comprising lidocaine, prilocaine, benzocaine, tetracaine, or combination thereof. The antiviral component may comprise one or more active ingredients selected from acyclovir, an acyclovir pro-drug such as valaciclovir, penciclovir, a penciclovir pro-drug such as famciclovir, or combinations thereof.

In one aspect, a compounded topical composition may include lidocaine in an amount between about 1% and about 4% by weight of the compounded topical composition; prilocaine in an amount between about 1% and about 4% by weight of the compounded topical composition; and an antiviral component between about 6% and about 10% by weight of the compounded topical composition. In one embodiment, the topical composition may comprise about 2.25% by weight lidocaine and prilocaine, respectively, and about 8% by weight acyclovir.

In another aspect, a compounded topical composition may include lidocaine in an amount between about 3% and about 6% by weight of the compounded topical composition and an antiviral component between about 6% and about 10% by weight of the compounded topical composition. In one embodiment, the topical composition may comprise about 4.5% by weight lidocaine and about 8% by weight acyclovir.

In yet another aspect, a compounded topical composition comprises an antiviral component comprising acyclovir, a local anesthetic component comprising lidocaine, and a topical base component comprising polyethylene glycol, wherein the compounded topical composition comprises between about 2% and about 4.75% lidocaine by weight and between about 4% and about 14% acyclovir by weight. In one embodiment, the compounded topical composition comprises about 4.25% lidocaine by weight and about 14% acyclovir by weight.

In one aspect, a compounded topical composition comprises an antiviral component comprising acyclovir, a local anesthetic component comprising lidocaine, and a topical base component comprising polyethylene glycol, wherein the compounded topical composition comprises between about 2% and about 5% lidocaine by weight and between about 4% and about 14% acyclovir by weight. In one embodiment, the compounded topical composition comprises about 3.5% lidocaine by weight and about 8% acyclovir by weight.

In another aspect, a compounded topical composition comprises an antiviral component comprising acyclovir, a local anesthetic component comprising lidocaine, and a topical base component comprising polyethylene glycol, wherein the compounded topical composition comprises between about 2% and about 4.75% lidocaine by weight and between about 2% and about 4.9% acyclovir by weight. In one embodiment, the compounded topical composition comprises about 2.5% lidocaine by weight and about 4.8% acyclovir by weight.

In various embodiments, the compounded topical composition includes a topical base composition including all or a portion of the topical base component and all or a portion of one or more active components each comprising one or more active ingredients thereof. For example, in some embodiments, the topical base composition includes all or a portion of both the topical base component and the antiviral component. In one formula, the topical base composition includes a commercially manufactured/available acyclovir topical, such as Acyclovir Ointment or Acyclovir Cream, to which the local anesthetic component and, in some embodiments, one or more additional active components are subsequently compounded. In some embodiments, the topical base composition includes all or a portion of both the topical base component and the local anesthetic component. For example, the topical bas composition may include a commercially manufactured/available Lidocaine or Lidocaine and Prilocaine Cream, such as Lidocaine 5% Ointment or Lidocaine and Prilocaine 2.5%/2.5% Cream, to which the antiviral component and, in some embodiments, one or more additional active components are subsequently compounded. Active components may comprise a fine powder of one or more active ingredients. The active ingredients in fine powder form may be generated from grinding up oral tablets containing the active ingredient. The active ingredients in fine powder form may be obtained from bulk powder. The active ingredients in fine powder form may be obtained from both bulk powder and generated from grinding up oral tablets containing the active ingredient. The fine powder may allow for precise amounts of the active ingredients to be combined with the topical base component. The compounded topical composition may exhibit excellent storage characteristics and avoid separation and/or degradation of the active ingredients from the base for substantial lengths of time. In some embodiments, one or more active ingredients may be combined with the topical base composition in the form of a solution, suspension, emulsion, or bulk powder.

In another aspect, a method of compounding one or more medications with a topical base component for the topical administration of a compounded topical composition therapy is provided. The method may include forming a fine powder of an antiviral component comprising grinding up one or more antiviral oral tablets and combining the antiviral component and the topical base component and local anesthetic component or a topical base composition comprising all or a portion of both the topical base component and the local anesthetic component. In one embodiment, the method may include combining a local anesthetic component and the topical base component. The local anesthetic component may be in the form of a solution, suspension, emulsion, bulk powder, or ground oral tablet containing on or more local anesthetics of the local anesthetic component. In one further example, a topical base composition includes the topical base component and the local anesthetic component or portions thereof. In one formulation, the topical base composition comprises a commercially manufactured/available topical composition including the local anesthetic component such as a Lidocaine and Prilocaine Cream. The method may also include combining the fine powder of active ingredients with a topical base composition containing lidocaine or both lidocaine and prilocaine, such that the compounded topical composition includes lidocaine or both lidocaine and prilocaine in an amount of between about 3% to about 6% by weight lidocaine or about 1% to about 4% of each of lidocaine and prilocaine by weight. In further embodiments, the method includes combining an additional active component selected from an anti-inflammatory component, an anticonvulsant component, a nerve depressant component, a muscle relaxant component, a NMDA (N-Methyl-D-aspartate) receptor antagonist component, an opiate or opioid agonist component, an antidepressant component, or combinations thereof. The method may include combining the fine powder of active ingredients with the topical base component in a sufficient amount such that the compounded topical composition includes the actives added in an amount of between about 10% and about 25% by weight of the compounded topical composition.

In one aspect, a method of producing a compounded topical composition comprises preparing a fine powder of an antiviral component comprising grinding one or more oral tablets of one or more antiviral actives to obtain a fine powder of the antiviral component. The antiviral component may comprise acyclovir. The method may further include combining the antiviral component with a topical base composition comprising a local anesthetic component and a topical base component. The local anesthetic component may be lidocaine and the topical base component may include polyethylene glycol. The topical base composition may be Lidocaine 5% Ointment. In one example, the antiviral component is combined with the topical base composition in an amount such that the compounded topical composition comprises between about 2% and about 4.75% lidocaine by weight and between about 4% and about 14% acyclovir by weight. In another example, the antiviral component is combined with the topical base composition in an amount such that the compounded topical composition comprises about 4.25% lidocaine by weight and about 14% acyclovir by weight.

In another aspect, a method of producing a compounded topical composition comprises preparing a fine powder of an antiviral component comprising grinding one or more oral tablets of one or more antiviral actives to obtain a fine powder of the antiviral component. The antiviral component may comprise acyclovir. The method may further include combining the antiviral component and a local anesthetic component with a topical base component comprising polyethylene glycol. The local anesthetic component may comprise lidocaine hydrochloride monohydrate. In one example, the antiviral component and the local anesthetic components are combined with the topical base component in amounts such that the compounded topical composition comprises between about 2% and about 5% lidocaine by weight and between about 4% and about 14% acyclovir by weight. In another example, the antiviral component and the local anesthetic components are combined with the topical base component in amounts such that the compounded topical composition comprises about 3.5% lidocaine by weight and about 8% acyclovir by weight.

In yet another aspect, a method of producing a compounded topical composition comprises combining a local anesthetic component comprising lidocaine hydrochloride monohydrate with a topical base composition, wherein the topical base composition comprises an antiviral component comprising acyclovir and a topical base component comprising polyethylene glycol. The topical base composition may comprise an Acyclovir 5% Ointment. In one example, the local anesthetic component is combined with the topical base composition in an amount such that the compounded topical composition comprises between about 2% and about 4.75% lidocaine by weight and between about 2% and about 4.9% acyclovir by weight. In another example, the local anesthetic component is combined with the topical base composition in an amount such that the compounded topical composition comprises about 2.5% lidocaine by weight and about 4.8% acyclovir by weight.

In one aspect, a method of producing a compounded topical composition comprises preparing a fine powder of an antiviral component comprising grinding one or more oral tablets of one or more antiviral actives to obtain a fine powder of the antiviral component; and combining the antiviral component and a topical base composition comprising a topical base component and a local anesthetic component and mixing the combined components. The antiviral component may be combined with the topical base composition comprising the topical base component and local anesthetic component in an amount such that the local anesthetic component is present in an amount between about 2% and about 8% by weight of the compounded topical composition and the antiviral component in an amount between 4% and 12% by weight of the compounded topical composition. The antiviral component may comprise one or more antiviral actives selected from acyclovir, valaciclovir, penciclovir, famciclovir, or combinations thereof, and the topical base composition may comprise a commercially manufactured/available lidocaine topical, a commercially manufactured/available lidocaine and prilocaine topical, or both. In some embodiments, the antiviral component may be (i) acyclovir, (ii) acyclovir and at least one of valaciclovir or famciclovir, (iii) valaciclovir, or (iv) famciclovir, wherein the topical base composition comprises Lidocaine 5% Ointment, Lidocaine and Prilocaine Cream, or combination thereof. In one example, the antiviral component may be acyclovir and combining the antiviral component and the topical base composition comprising the topical base component and the local anesthetic component comprises combining a sufficient amount of fine powder of acyclovir prepared from the grinding of oral tablets of acyclovir to obtain between about 6% and about 9% acyclovir by weight of the compounded topical composition. The topical base composition may comprise Lidocaine 5% Ointment, Lidocaine and Prilocaine Cream, or combination thereof.

In another example, the antiviral component comprises acyclovir and combining the antiviral component and the topical base composition comprising the topical base component and the local anesthetic component comprises combining a sufficient amount of fine powder of acyclovir prepared from the grinding of oral tablets of acyclovir to obtain about 8% acyclovir by weight of the compounded topical composition. The topical base composition may comprise Lidocaine 5% Ointment, Lidocaine and Prilocaine Cream, or combination thereof.

In yet another example, the antiviral component comprises acyclovir and the topical base composition comprises Lidocaine 5% Ointment, wherein combining the antiviral component and the Lidocaine 5% Ointment comprises combining a sufficient amount of fine powder of acyclovir prepared from the grinding of oral tablets of acyclovir to obtain between about 6% and about 9% acyclovir and between about 4% and about 5% lidocaine by weight of the compounded topical composition.

In still yet another example, the antiviral component comprises acyclovir and the topical base composition comprises Lidocaine 5% Ointment, wherein combining the antiviral component and the Lidocaine 5% Ointment comprises combining a sufficient amount of fine powder of acyclovir prepared from the grinding of oral tablets of acyclovir to obtain about 8% acyclovir and about 4.5% lidocaine by weight of the compounded topical composition.

In one example, the antiviral component comprises acyclovir and at least one of valaciclovir or famciclovir and the topical base composition comprises Lidocaine 5% Ointment, wherein combining the antiviral component and the Lidocaine 5% Ointment comprises combining a sufficient amount of fine powder of acyclovir and at least one of valaciclovir or famciclovir prepared from the grinding of oral tablets of the acyclovir and the at least one of valaciclovir or famciclovir to obtain between about 6% and about 9% acyclovir and the at least one of valaciclovir or famciclovir by weight of the compounded topical composition and between about 4% and about 5% lidocaine by weight of the compounded topical composition.

In another example, the antiviral component comprises acyclovir and at least one of valaciclovir or famciclovir and the topical base composition comprises Lidocaine 5% Ointment, wherein combining the antiviral component and the Lidocaine 5% Ointment comprises combining a sufficient amount of fine powder of acyclovir and the at least one of valaciclovir or famciclovir prepared from the grinding of oral tablets of acyclovir and the at least one of valaciclovir or famciclovir to obtain about 8% acyclovir and the at least one of valaciclovir or famciclovir by weight of the compounded topical composition and about 4.5% lidocaine by weight of the compounded topical composition.

In yet another example, the antiviral component comprises at least one of valaciclovir or famciclovir and the topical base composition comprises Lidocaine 5% Ointment, wherein combining the antiviral component and the Lidocaine 5% Ointment comprises combining a sufficient amount of fine powder of the at least one of valaciclovir or famciclovir prepared from the grinding of oral tablets of the at least one of valaciclovir or famciclovir to obtain between about 6% and about 9% of the valaciclovir or famciclovir by weight of the compounded topical composition and between about 4% and about 5% lidocaine by weight of the compounded topical composition.

In still yet another example, the antiviral component comprises at least one of valaciclovir or famciclovir and the topical base composition comprises Lidocaine 5% Ointment, wherein combining the antiviral component and the Lidocaine 5% Ointment comprises combining a sufficient amount of fine powder of the at least one of valaciclovir or famciclovir prepared from the grinding of oral tablets of the at least one of valaciclovir or famciclovir to obtain about 8% of the at least one of valaciclovir or famciclovir by weight of the compounded topical composition and about 4.5% lidocaine by weight of the compounded topical composition.

In one example, the antiviral component comprises acyclovir and the topical base composition comprises Lidocaine and Prilocaine 2.5%/2.5% Cream, wherein combining the antiviral component and the Lidocaine and Prilocaine 2.5%/2.5% Cream comprises combining a sufficient amount of fine powder of acyclovir prepared from the grinding of oral tablets of acyclovir to obtain between about 6% and about 9% acyclovir and between about 1.5% and about 2.25% of both lidocaine and prilocaine by weight of the compounded topical composition.

In another example, the antiviral component comprises acyclovir and the topical base composition comprises Lidocaine and Prilocaine 2.5%/2.5% Cream, wherein combining the antiviral component and the Lidocaine and Prilocaine 2.5%/2.5% Cream comprises combining a sufficient amount of fine powder of acyclovir prepared from the grinding of oral tablets of acyclovir to obtain about 8% acyclovir and about 2.5% of both lidocaine and prilocaine by weight of the compounded topical composition.

In yet another example, the antiviral component comprises acyclovir and at least one of valaciclovir or famciclovir and the topical base composition comprises Lidocaine and Prilocaine 2.5%/2.5% Cream, wherein combining the antiviral component and the Lidocaine and Prilocaine 2.5%/2.5% Cream comprises combining a sufficient amount of fine powder of acyclovir and the at least one of valaciclovir or famciclovir prepared from the grinding of oral tablets of acyclovir and the at least one of valaciclovir or famciclovir to obtain between about 6% and about 9% acyclovir and the at least one of valaciclovir or famciclovir by weight of the compounded topical composition and between about 1.5% and about 2.25% of both lidocaine and prilocaine by weight of the compounded topical composition.

In still yet another example, the antiviral component comprises acyclovir and at least one of valaciclovir or famciclovir and the topical base composition comprises Lidocaine and Prilocaine 2.5%/2.5% Cream, wherein combining the antiviral component and the Lidocaine and Prilocaine 2.5%/2.5% Cream comprises combining a sufficient amount of fine powder of acyclovir and the at least one of valaciclovir or famciclovir prepared from the grinding of oral tablets of acyclovir and the at least one of valaciclovir or famciclovir to obtain about 8% acyclovir and the at least one of valaciclovir or famciclovir by weight of the compounded topical composition and about 2.5% of both lidocaine and prilocaine by weight of the compounded topical composition.

In one example, the antiviral component comprises at least one of valaciclovir or famciclovir and the topical base composition comprises Lidocaine and Prilocaine 2.5%/2.5% Cream, wherein combining the antiviral component and the Lidocaine and Prilocaine 2.5%/2.5% Cream comprises combining a sufficient amount of fine powder of the at least one of valaciclovir or famciclovir prepared from the grinding of oral tablets of the at least one of valaciclovir or famciclovir to obtain between about 6% and about 9% of the at least one of valaciclovir or famciclovir by weight of the compounded topical composition and between about 1.5% and about 2.25% of both lidocaine and prilocaine by weight of the compounded topical composition.

In another example, the antiviral component comprises at least one of valaciclovir or famciclovir and the topical base composition comprises Lidocaine and Prilocaine 2.5%/2.5% Cream, wherein combining the antiviral component and the Lidocaine and Prilocaine 2.5%/2.5% Cream comprises combining a sufficient amount of fine powder of the at least one of valaciclovir or famciclovir prepared from the grinding of oral tablets of the at least one of valaciclovir or famciclovir to obtain about 8% of the at least one of valaciclovir or famciclovir by weight of the compounded topical composition and about 2.5% of both lidocaine and prilocaine by weight of the compounded topical composition.

The above-described and other features and advantages of the present disclosure will be appreciated and understood by those skilled in the art from the following detailed description and appended claims.

### DETAILED DESCRIPTION

The present embodiments may relate to a topically deliverable compounded topical composition for treatment of one or more viral ailments related to herpes simplex and varicella zoster, including herpes simplex virus type I (HSV-1) and herpes simplex virus type II (HSV-2). Various embodiments of the compounded topical composition may be formulated and used to prevent or reduce the risk acquisition, spread, or flare-ups/outbreaks of herpes simplex infections, including, but not limited to, labialis, genital, and neonatal infections. The compounded topical composition may be further formulated and used to prevent or reduce the risk of acquisition, spread, or flare-ups/outbreaks of varicella zoster infections, such as chickenpox and shingles. In some embodiments, the topically deliverable compounded topical composition may be formulated and used for treatment of one or more viral ailments related to Epstein-Barr virus and cytomegalovirus. Some embodiments may find use in the treatment or prevention of other viral or microbial infections.

### I. Compounded Topical Compositions

Compounded topical compositions described herein may include a topical base component and one or more active components combined with, e.g., added and mixed with, the topical base component. Unless indicated otherwise, topical may include formulation for administration to a body surface, such as skin or mucus membrane, and may include local delivery of one or more actives, systemic delivery of one or more actives, or both. Topical may also include formulation for transdermal or transmucosal delivery.

Active components of the compounded topical composition may include an antiviral component and a local anesthetic component. Each active component of the compounded tropical cream may include one or more actives. The local anesthetic component may comprise one or more actives selected from lidocaine, benzocaine, prilocaine, tetracaine, or a combinations thereof. The antiviral component may comprises one or more actives selected from acyclovir, an acyclovir pro-drug such as valaciclovir, penciclovir, a penciclovir pro-drug such as famciclovir, or combinations thereof.

In various embodiments, the compounded topical composition comprises a topical base component, a local anesthetic component, and an antiviral component, and formulating the compounded topical composition comprises combining the antiviral component with the topical base component and the local anesthetic component or with a topical base composition comprising both the topical base component and the local anesthetic component. In some embodiments, the compounded topical composition comprises a topical base component, a local anesthetic component, and an antiviral component, and formulating the compounded topical composition comprises combining the local anesthetic component with the topical base component and the antiviral component or a topical base composition comprising both the topical base component and the antiviral component.

The antiviral component may comprise one or more actives selected from acyclovir, an acyclovir pro-drug such as valaciclovir, penciclovir, a penciclovir pro-drug such as famciclovir, or combinations thereof. The local anesthetic component may comprise one or more actives selected from lidocaine, prilocaine, benzocaine, tetracaine, or combinations thereof. The topical base component may include a colloid, emulsion, foam, gel or jelly, cream, ointment, lotion, powder, solution, suspension, spray, aerosol, or other suitable topical base format to deliver the active components. Unless specified otherwise, cream may be used herein to generally refer to a topical base composition that is a colloid, emulsion, foam, gel or jelly, cream, ointment, or lotion. In various embodiments, the topical base component may include polyethylene glycol (PEG), petroleum, vegetable oils, paraffin, waxes, silicones, or suitable base vehicle or combinations of base vehicles. In one embodiment, the topical base component includes a compounded topical anhydrous silicone containing pracaxi oil. Silicones or silicone variants may be present in amounts between about 20% and about 70% by weight of the topical base component. In one such example, the topical base component comprises an anhydrous silicone gel distributed by Professional Compounding Centers of America (PCCA), 9901 S. Wilcrest Dr, Houston, Tex. 77099, sold under the name PCCA PracaSil™-Plus, which contains pracaxi oil and over 50% silicones. In some embodiments, the topical base component may include or be provided in a topical base composition with one or more active components or one or more active ingredients. For example, the topical base component may include or be provided in a topical base composition comprising a commercially manufactured/available antiviral or local anesthetic cream, which may be an ointment.

In various embodiments, the topical base component and all or a portion of the local anesthetic component may be provided in a topical base composition. For example, a topical base composition may include local anesthetic component together with the topical base component in a Lidocaine and Prilocaine Cream to which the antiviral component and, in some embodiments, one or more additional active components may be added. In some embodiments, the topical base composition includes a commercially manufactured/available transdermal format that includes the topical base component and all or a portion of the local anesthetic component. For example, the topical base composition may comprise a lidocaine topical, e.g., Commercially manufactured/availabletment, or a lidocaine and prilocaine topical, e.g., Lidocaine and Prilocaine Cream. The Lidocaine and Prilocaine Cream may be a commercially manufactured/available Lidocaine and Prilocaine Cream. For example, the topical base composition may include Lidocaine and Prilocaine Cream (e.g., EMLA cream or generic), Lidocaine Cream (e.g., Commercially manufactured/availabletment), or other composition comprising lidocaine, prilocaine, or both, to topically deliver lidocaine, prilocaine, or both at skin or mucous membrane. In one example, a commercially manufactured/available Lidocaine and Prilocaine Cream comprises lidocaine, prilocaine, polyoxyethylene fatty acid esters, sodium hydroxide, purified water, and at least one of carboxypolymethylene or carbomer 934. Example commercially manufactured/available Lidocaine and Prilocaine Creams include EMLA® Cream (Lidocaine 2.5% and Prilocaine 2.5%) marketed by Akorn Pharmaceuticals, Lake Forest, Ill. or ACTAVIS, Parsippany, NJ or generic Lidocaine and Prilocaine Cream 2.5%/2.5%, such as Lidocaine 2.5% and Prilocaine 2.5% Cream, manufactured by Hi-Tech Pharmacal Co., Inc. Amityville, NY and Lidocaine and Prilocaine Cream 2.5%/2.5%, marketed by E. Fougera & Co., a division of Fougera Pharmaceuticals Inc., Melville, New York. In another example, a commercially manufactured/available Lidocaine Cream/Ointment includes a water miscible ointment vehicle containing polyethylene glycols (PEGs). An example lidocaine cream is Commercially manufactured/availabletment 5% marketed by E. Fougera and Co., a division of Fougera Pharmaceuticals Inc., Melville, New York, which includes acetamide, 2-(diethylamino)-N-(2,6-dimethylphenyl)-, (lidocaine) 5% in a water miscible ointment vehicle containing polyethylene glycols.

In one embodiment, the compounded topical composition comprises Acyclovir 5% Ointment and at least one of Lidocaine 5% Ointment or Lidocaine and Prilocaine 2.5%/2.5% Cream.

In some embodiments, the compounded topical composition comprises one or more additional active components selected from an anti-inflammatory component, an anticonvulsant component, a nerve depressant component, a muscle relaxant component, a NMDA (N-Methyl-D-aspartate) receptor antagonist component, an opiate or opioid agonist component, an antidepressant component, or combinations thereof.

The anti-inflammatory component may include one or more NSAIDS (non-steroidal anti-inflammatory drug) selected from (1) oxicams such as meloxicam or piroxicam; (2) salicylic acid derivatives such as aspirin, diflunisal, salsalate, or trilisate; (3) propionic acids such as flurbiprofen, ibuprofen, ketoprofen, naproxen, or oxaprozin; (4) acetic acids such as diclofenac, etodolac, indomethacin, ketorolac, nabumetone, sulindac, or tolmetin; (5) fenamates such as meclofenamate; (6) COX-2 inhibitors such as celecoxib, rofecoxib, and valdecoxib; or (7) combinations thereof. The anticonvulsant or nerve depressant component may comprise one or more nerve depressants and/or anticonvulsants selected from gabapentin, topiramate, lamotrigine, or combinations thereof. The muscle relaxant component may include one or more actives selected baclofen, carisoprodol, chlorzoxazone, cyclobenzaprine, dantrolene, diazepam, metaxalone, methocarbamol, orphenadrine, quinine sulfate, tizanidine, and/or other muscle relaxants. The NMDA receptor antagonist component may include ketamine. The opiate or opioid agonist component may comprise one or more actives selected from tramadol; one or more C2 opiate agonists selected from oxycodone, morphine, methadone, hydromorphone, and fentanyl; one or more C3 opiate agonists selected from hydrocodone, codeine, propoxyphene, butalbital, and pentazocine; or any combination thereof.

In some embodiments, one or more active components or one or more actives thereof may be combined with the topical base component or topical base composition in the form of a solution, suspension, emulsion, or bulk powder.

In various embodiments, one or more of the active components or one or more actives thereof may be in the form of powders combined with the topical base component or topical base composition that includes all or a portion of the topical base component and all or a portion of an active component. The fine powders may be combined with the topical base component or topical base composition and mixed to form the compounded topical composition. Combining may include adding components or portions thereof in any order.

In one embodiment, the compounded topical composition includes lidocaine in an amount between about 0.5% and about 4.0% by weight of the compounded topical composition; prilocaine in an amount between about 0.5% and about 4.0% by weight of the compounded topical composition; and an antiviral component between about 4.0% and about 15% by weight of the compounded topical composition, wherein the antiviral component is or comprises acyclovir or one or more actives selected from acyclovir, valaciclovir, penciclovir, famciclovir, or combinations thereof.

In another embodiment, the compounded topical composition includes lidocaine in an amount between about 2% and about 6% by weight of the compounded topical composition and an antiviral component between about 4% and about 15% by weight of the compounded topical composition, wherein the antiviral component is or comprises acyclovir or one or more actives selected from acyclovir, valaciclovir, penciclovir, famciclovir, or combinations thereof. In one such example, the compounded topical composition does not include prilocaine.

In still another embodiment, the compounded topical composition includes lidocaine in an amount between about 0.5% and about 4.0% by weight of the compounded topical composition; prilocaine in an amount between about 0.5% and about 4.0% by weight of the compounded topical composition; and an antiviral component between about 4.0% and about 15% by weight of the compounded topical composition, wherein the antiviral component is or comprises acyclovir or one or more actives selected from acyclovir, valaciclovir, penciclovir, famciclovir, or combinations thereof.

In yet another embodiment, the compounded topical composition includes lidocaine in an amount between about 2% and about 6% by weight of the compounded topical composition and an antiviral component between about 4% and about 15% by weight of the compounded topical composition, wherein the antiviral component is or comprises acyclovir or one or more actives selected from acyclovir, valaciclovir, penciclovir, famciclovir, or combinations thereof. In one such example, the compounded topical composition does not include prilocaine.

In one embodiment, the compounded topical composition includes the local anesthetic component in an amount between about 2% and about 8% by weight of the compounded topical composition and the antiviral component in an amount between about 2% and about 14% by weight of the compounded topical composition. In one example, the local anesthetic component includes lidocaine and prilocaine and the antiviral component comprises or is acyclovir or one or more actives selected from acyclovir, valaciclovir, penciclovir, famciclovir, or combinations thereof. In one example of the embodiment, the local anesthetic component includes lidocaine and prilocaine, both in an amount of at least about 1%, about 1.5%, about 2%, about 2.5%, about 3%, about 3.5%, or about 4% by weight of the compounded topical composition and the antiviral component comprises or is acyclovir in an amount of about 2%, about 2.5%, about 3%, about 3.5%, about 4%, about 4.5%, about 5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, or about 14% by weight of the compounded topical composition. In another example, the antiviral component is selected from one or more actives comprising valaciclovir, penciclovir, famciclovir, or combinations thereof, in an amount of about 2%, about 2.5%, about 3%, about 3.5%, about 4%, about 4.5%, about 5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, or about 14% by weight of the compounded topical composition. In another example, the antiviral component includes acyclovir in an amount about 4%, about 4.5%, about 5%, about 6%, about 6.5%, about 7%, about 7.5%, or about 8% by weight of the compounded topical composition and at least one additional active selected from valaciclovir, penciclovir, famciclovir, or combinations thereof, in an amount of about 1%, about 1.5%, about 2%, about 2.5%, about 3%, about 3.5%, or about 4% by weight of the compounded topical composition. In yet another example, the antiviral component comprises or is valaciclovir in an amount of about 2%, about 2.5%, about 3%, about 3.5%, about 4%, about 4.5%, about 5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, or about 14% by weight of the compounded topical composition. In yet still another example, the antiviral component comprises penciclovir, famciclovir, or a combination thereof, in an amount of about 2%, about 2.5%, about 3%, about 3.5%, about 4%, about 4.5%, about 5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, or about 14% by weight of the compounded topical composition. In another example, the local anesthetic component includes lidocaine and prilocaine, both in an amount between about 1.5% and about 2.5%, about 2% and about 2.5%, or about 2.25% by weight of the compounded topical composition and the antiviral component comprises or is acyclovir in an amount between about 5% and about 10%, about 6% and about 9%, about 7% and about 9%, or about 8% by weight of the compounded topical composition. In one example, the antiviral component is selected from one or more actives comprising valaciclovir, penciclovir, famciclovir, or combinations thereof, in an amount between about 5% and about 10%, about 6% and about 9%, about 7% and about 9%, or about 8% by weight of the compounded topical composition. In another example, the antiviral component includes acyclovir in an amount between about 2% and about 8%, about 2% and about 6%, about 2% and about 4%, or about 6% by weight of the compounded topical composition and at least one additional active selected from valaciclovir, penciclovir, famciclovir, or combinations thereof, in an amount between about 1% and about 8%, about 2% and about 6%, about 3% and 5%, about 4% and about 6%, or about 3% by weight of the compounded topical composition. In yet another example, the antiviral component comprises or is valaciclovir in an amount between about 5% and about 10%, about 6% and about 9%, about 7% and about 9%, or about 8% by weight of the compounded topical composition. In yet still another example, the antiviral component comprises penciclovir, famciclovir, or a combination thereof, in an amount between about 5% and about 10%, about 6% and about 9%, about 7% and about 9%, or about 8% by weight of the compounded topical composition.

In one embodiment, the compounded topical composition includes the local anesthetic component in an amount between about 2% and about 8% by weight of the compounded topical composition and the antiviral component in an amount between about 2% and about 14% by weight of the compounded topical composition wherein the local anesthetic component comprises lidocaine and little or no prilocaine. In one example, the antiviral component is acyclovir or one or more actives selected from acyclovir, valaciclovir, penciclovir, famciclovir, or combinations thereof. In a further example, the anesthetic component is lidocaine and the compounded topical composition does not include prilocaine. In one example of the embodiment, the local anesthetic component is or includes lidocaine and the antiviral component is or comprises acyclovir or one or more actives selected from acyclovir, valaciclovir, penciclovir, famciclovir, or combinations thereof. In other such examples, the local anesthetic component comprises or is lidocaine and the antiviral component comprises or is acyclovir or one or more actives selected from acyclovir, valaciclovir, penciclovir, famciclovir, or combinations thereof. In one example, the local anesthetic component comprises or is lidocaine in an amount of at least about 1%, about 1.5%, about 2%, about 2.5%, about 3%, about 3.5%, about 4%, about 4.5%, about 5%, about 5.5%, about 6%, about 6.5%, about 7%, about 7.5%, or about 8% by weight of the compounded topical composition and the antiviral component comprises or is acyclovir in an amount of about 2%, about 2.5%, about 3%, about 3.5%, about 4%, about 4.5%, about 5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, or about 14% by weight of the compounded topical composition. In another example, the antiviral component is selected from one or more actives comprising valaciclovir, penciclovir, famciclovir, or combinations thereof, in an amount of about 2%, about 2.5%, about 3%, about 3.5%, about 4%, about 4.5%, about 5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, or about 14% by weight of the compounded topical composition. In another example, the antiviral component comprises acyclovir in an amount about 4%, about 4.5%, about 5%, about 6%, about 6.5%, about 7%, about 7.5%, or about 8% by weight of the compounded topical composition and at least one additional active selected from valaciclovir, penciclovir, famciclovir, or combinations thereof, in an amount of about 1%, about 1.5%, about 2%, about 2.5%, about 3%, about 3.5%, or about 4% by weight of the compounded topical composition. In yet another example, the antiviral component comprises or is valaciclovir in an amount of about 2%, about 2.5%, about 3%, about 3.5%, about 4%, about 4.5%, about 5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, or about 14% by weight of the compounded topical composition. In yet still another example, the antiviral component comprises penciclovir, famciclovir, or a combination thereof, in an amount of about 2%, about 2.5%, about 3%, about 3.5%, about 4%, about 4.5%, about 5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, or about 14% by weight of the compounded topical composition. In another example, the local anesthetic component is lidocaine in an amount between about 2% and about 8%, about 3% and about 7%, about 4% and about 6%, about 4% and about 5%, or about 4.5% by weight of the compounded topical composition and the antiviral component includes acyclovir in an amount between about 5% and about 12%, about 5% and about 10%, about 6% and about 9%, about 7% and about 9%, or about 8% by weight of the compounded topical composition. In one example, the antiviral component is selected from one or more actives comprising valaciclovir, penciclovir, famciclovir, or combinations thereof, in an amount between about 5% and about 12%, about 5% and about 10%, about 6% and about 9%, about 7% and about 9%, or about 8% by weight of the compounded topical composition. In another example, the antiviral component includes acyclovir in an amount between about 2% and about 8%, about 2% and about 6%, about 2% and about 4%, or about 6% by weight of the compounded topical composition and at least one additional active selected from valaciclovir, penciclovir, famciclovir, or combinations thereof, in an amount between about 1% and about 8%, about 2% and about 6%, about 3% and 5%, about 4% and about 6%, or about 3% by weight of the compounded topical composition. In yet another example, the antiviral component comprises or is valaciclovir in an amount between about 5% and about 10%, about 6% and about 9%, about 7% and about 9%, or about 8% by weight of the compounded topical composition. In yet still another example, the antiviral component comprises penciclovir, famciclovir, or a combination thereof, in an amount between about 5% and about 12%, about 5% and about 10%, about 6% and about 9%, about 7% and about 9%, or about 8% by weight of the compounded topical composition.

In one embodiment, the compounded topical composition comprises about 2.25% by weight lidocaine and prilocaine, respectively, and about 8% by weight acyclovir. In another embodiment, the topical composition comprises about 4.5% by weight lidocaine and about 8% by weight acyclovir. In one such embodiment, the compounded composition does not include prilocaine. In one embodiment, the local anesthetic component comprises or is lidocaine in an amount of at least about 3.5% by weight of the compounded topical composition and the antiviral component comprises or is acyclovir in an amount of about 3.5% by weight of the compounded topical composition. In one embodiment, the local anesthetic component comprises or is lidocaine in an amount of at least about 2.5% by weight of the compounded topical composition and the antiviral component comprises or is acyclovir in an amount of about 4.8% by weight of the compounded topical composition. In one embodiment, the local anesthetic component comprises or is lidocaine in an amount of at least about 4.25% by weight of the compounded topical composition and the antiviral component comprises or is acyclovir in an amount of about 12% by weight of the compounded topical composition.

In any of the above embodiments, the compound topical composition may include one or more additional active components. In one example, the compounded topical composition includes an anti-inflammatory component comprising one or more actives, such as flurbiprofen or nabumetone, in an amount between about 5.0% and about 25.0% of the topical composition by weight. In this or another example, the compounded topical composition includes a nerve depressant component comprising one or more actives, such as gabapentin, in an amount between about 5.0% and about 15.0% of the compounded topical composition by weight. In either of the above or another example, the compounded topical composition includes a muscle relaxant component comprising one or more actives, such as cyclobenzaprine, in an amount between about 0.5% and about 4.0% by weight of the compounded topical composition. In one embodiment, the compounded topical composition may comprise about 2.0% lidocaine, about 2.0% prilocaine, about 4.5% lidocaine, about 8% acyclovir and at least one of about 4% to 6% gabapentin, about 0.5% to 1.0% cyclobenzaprine, and about 5% to about 10% flurbiprofen or about 10% to about 20% nabumetone. In one embodiment, acyclovir is present in the compounded topical composition in an amount about 1% to about 10%, about 4% to about 9%, or about 5% to about 8% by weight and lidocaine or lidocaine and prilocaine are present in the compounded topical composition an amount about 4% to about 5% by weight. Additional active components may also be present such as about 1% to about 10% gabapentin, about 1% to about 10%, about 2% to about 8%, about 3% to about 7%, or about 4% to about 6% by weight diclofenac, about 0.5% to about 2%, about 0.5% to about 1.5% by weight cyclobenzaprine.

### II. Methods of Compounding

A method of compounding one or more active medications with a topical base component for the topical administration of a compounded topical composition is provided.

The method may include combining one or more active components or actives thereof and the topical base component (which may include a topical base composition comprising all or a portion of the topical base component and all or a portion of an active component). Combining may comprise mixing, which may include milling in an ointment mill. One or more of the active components or actives thereof may be combined with the topical base component (or topical base composition comprising all or a portion of the topical base component and all or a portion of the active component or another active component) as a solution, suspension, colloid, emulsion, bulk powder, or ground oral tablet. Thus, the method may include preparing one or more active components or one or more actives thereof for addition to the topical base component. In one example, preparing the one or more active components or one or more actives thereof for addition to the topical base component includes grinding oral tablets comprising the actives. In this or another example, preparing the one or more active components or one or more actives thereof for addition to the topical base component comprises dissolving, solubilizing, wetting, dispersing, or suspending one or more actives in solution. The actives may be ground oral tablets or bulk powder. Dissolving, solubilizing, wetting, dispersing, or suspending may include combining the one or more actives with water for injection or irrigation, DMSO, alcohol, or other suitable liquid. When a topical base composition comprising a portion of the topical base component or portion of an active component, the remaining portion of the topical base component or portion of the active component may be combined in the form of a colloid, emulsion, foam, gel or jelly, cream, ointment, lotion, powder (ground from tablets or bulk), solution, suspension, spray, aerosol, or other suitable format.

In one embodiment, the method includes forming a fine powder of an antiviral component comprising grinding up one or more antiviral oral tablets and combining the antiviral component and the topical base component. In this or another embodiment, the method may include combining the local anesthetic component and the topical base component. The local anesthetic component may be in the form of a solution, suspension, emulsion, bulk powder, or find ground oral tablet containing on or more local anesthetics of the local anesthetic component. Thus, the method may include grinding oral tablets of one or more local anesthetic actives. In a further example, a topical base composition comprises all or a portion of the topical base component and local anesthetic component. In one formulation, for example, the topical base composition comprises a commercially manufactured/available transdermal cream including the local anesthetic component such as a Lidocaine Cream or Lidocaine and Prilocaine Cream, which may include a gel, ointment, or emulsion. The fine powder of active ingredients may be combined with the topical base composition containing lidocaine or both lidocaine and prilocaine, such that the compounded topical composition includes lidocaine or both lidocaine and prilocaine in an amount by weight of the compounded topical composition described above.

A method of compounding may include providing a topical base composition having the topical base component and one or more local anesthetics; and combining with the base a fine powder of medication comprising one or more antiviral actives of the antiviral component. In a further example, the method may further include combining a fine powder of actives of one or more additional components. The one or more additional components may be selected from an anti-inflammatory component, an anticonvulsant component, a nerve depressant component, a muscle relaxant component, a NMDA (N-Methyl-D-aspartate) receptor antagonist component, an opiate or opioid agonist component, an antidepressant component, or combinations thereof. One, more, of all actives of the additional components may be combined with the topical base composition in the form of a solution, suspension, emulsion, bulk powder, or ground commercial tablet. In one such embodiment, the method may include preparing a one or more of the actives of the additional components for addition to the topical base composition comprising grinding commercial tablets comprising the actives to form a fine powder.

As introduced above, the active ingredients in fine powder form may be generated from grinding up oral tablets containing the active ingredient. The fine powder may allow for precise amounts of the active ingredients to be combined with the base. The compounded topical composition may exhibit excellent storage characteristics, and avoid separation and/or degradation of the active ingredients from the base for substantial lengths of time. According to various embodiments, the compounded composition formulated, prepared, and packaged as described here include stability suitable for labeling of USP 795 non-aqueous 180 days beyond use date.

In one formulation of the method, the topical base composition comprises a commercially manufactured/available Lidocaine and Prilocaine 2.5%/2.5% Cream, a commercially manufactured/available Lidocaine 5% Ointment, or a combination of both and the antiviral component comprises a powder generated from grinding commercially manufactured and commercially available oral tablets comprising the antiviral component or one or more actives thereof. In another formulation, the local anesthetic component comprising lidocaine or lidocaine and prilocaine may be combined with to the topical base component before or after the antiviral component is combined with the topical base component rather than using a topical base composition that already includes the local anesthetic component. When lidocaine or lidocaine and prilocaine are combined with the topical base component, the lidocaine or lidocaine and prilocaine may be in the form of a solution, suspension, emulsion, gel, cream, ointment, powder (which may be a bulk powder or fine ground powder obtained from ground oral tablets), for example. Prilocaine may also be combined with a topical base composition including lidocaine and the topical base component, such as lidocaine gel, cream, ointment, or lotion.

In various embodiments of the method, the topical base composition includes all or a portion of the topical base component and all or a portion of the antiviral component. For example, the antiviral component may be provided together with the topical base component in an antiviral topical cream to which the local anesthetic component and, in some embodiments, one or more additional active components are subsequently compounded. In some embodiments, the topical base composition includes a commercially manufactured/available transdermal format that includes all or a portion of both the topical base component and the antiviral component. For example, the topical base composition may comprise an acyclovir topical and thus be provided together with all or a portion of the antiviral component. The topical base composition may include an acyclovir topical to which additional compatible topical base may be added, e.g., to reduce the potency of the antiviral in the final compounded topical composition, provide emollient functions, or modify characteristics of the final compounded topical composition, for example. The acyclovir topical preferably includes an Acyclovir Ointment; however, in some embodiments, the acyclovir topical may be or include an Acyclovir Cream. One example acyclovir topical is Acyclovir 5% Ointment. Acyclovir 5% Ointment is approved by the U.S. Food and Drug Administration (FDA) and contains 50 mg of acyclovir in a polyethylene glycol (PEG) base per gram and is supplied in 30 g tubes. Acyclovir 5% Ointment was initially manufactured and distributed under the name Zovirax Ointment 5%; however, Acyclovir 5% Ointment, is currently manufactured in generic form. Zovirax Cream 5% contains 50 mg of acyclovir and the following inactive ingredients: cetostearyl alcohol, mineral oil, poloxamer 407, propylene glycol, sodium lauryl sulfate, water, and white petrolatum and has been supplied in 2 g and 5 g tubes. Other compatible acyclovir topicals in PEG bases may be used, including greater or lesser strength formulations as they become available.

In one formulation, the topical base composition comprises a commercially manufactured/available Lidocaine and Prilocaine 2.5%/2.5% Cream, a commercially manufactured/available Lidocaine 5% Ointment, or a combination of both and the antiviral component comprises a powder generated from grinding commercially manufactured/available oral tablets comprising the antiviral. In another formulation, the local anesthetic component comprising lidocaine or lidocaine and prilocaine may be combined with to the topical base component before or after the antiviral component is combined with the topical base component rather than using a topical base composition that already includes the local anesthetic component. When lidocaine or lidocaine and prilocaine are combined with the topical base component, the lidocaine or lidocaine and prilocaine may be in the form of a solution, suspension, emulsion, gel, cream, ointment, powder (which may be a bulk powder or fine ground powder obtained from ground oral tablets), for example. Prilocaine may also be combined with a lidocaine topical base component, such as lidocaine gel, cream, ointment, or lotion.

In various embodiments of the method, the antiviral component and a topical base composition comprising all or a portion of both the topical base component and the local anesthetic component may be combined in amounts such that the compounded topical composition comprises the antiviral component in an amount between about 4% and about 12% by weight of the compounded topical composition and the local anesthetic component in about equivalent amounts of each of lidocaine and prilocaine between about 1.5% and about 2.5% by weight of the compounded topical composition. In another embodiment, the antiviral component and the topical base composition may be combined in amounts such that the compounded topical composition comprises the antiviral component in an amount between about 4% and about 12% by weight of the compounded topical composition and the local anesthetic component in an amount of lidocaine between about 2.5% and about 5% by weight of the compounded topical composition. In another embodiment, the antiviral component and the topical base composition may be combined in amounts such that the compounded topical composition comprises the antiviral component in an amount between about 4% and about 14% by weight of the compounded topical composition and the local anesthetic component in an amount of lidocaine between about 2% and about 5% by weight of the compounded topical composition.

The method may comprise providing a topical base composition comprising the topical base component and one or more local anesthetic actives of the local anesthetic component. Primary examples of local anesthetics that the topical compositions and base composition disclosed herein may employ include, but are not limited to, lidocaine, prilocaine, benzocaine, and/or tetracaine. In some embodiments, the topical base composition includes a commercially manufactured/available transdermal format that includes all or a portion of the topical base component and all or a portion of the local anesthetic component. For example, the topical base composition may include Lidocaine and Prilocaine Cream (e.g., EMLA cream or generic), lidocaine cream, or other topical base composition comprising lidocaine, prilocaine, or both, formulated to topically deliver lidocaine, prilocaine, or both at skin or mucous membrane. In one embodiment, the local anesthetic component may comprise between about 2% and about 6.0% by weight of the compounded topical composition. Other amounts may be used, including those discussed elsewhere herein. The topical base composition may include additional, fewer, or alternate ingredients. In one embodiment, the topical base composition includes lidocaine and/or prilocaine. In one example, the topical base composition comprises an equal amount of lidocaine and prilocaine, such as between about 2.0% and about 3.0% by weight. In one embodiment, the topical base composition is or includes commercially manufactured/availabletment, such as Lidocaine 5% Ointment. Other amounts or strengths may be used, including those discussed elsewhere herein.

In various embodiments of the method, the local anesthetic component and a topical base composition comprising all or a portion of both the topical base component and the antiviral component may be combined in amounts such that the compounded topical composition comprises the antiviral component in an amount between about 4% and about 12% by weight of the compounded topical composition and the local anesthetic component in about equivalent amounts of each of lidocaine and prilocaine between about 1.5% and about 2.5% by weight of the compounded topical composition. In another embodiment, the local anesthetic component and the topical base composition may be combined in amounts such that the compounded topical composition comprises the antiviral component in an amount between about 4% and about 12% by weight of the compounded topical composition and the local anesthetic component in an amount of between about 2.5% and about 5% by weight of the compounded topical composition wherein the antiviral component comprises acyclovir and the local anesthetic component is lidocaine. In another embodiment, the local anesthetic component and the topical base composition may be combined in amounts such that the compounded topical composition comprises the antiviral component in an amount between about 4% and about 15% by weight of the compounded topical composition and the local anesthetic component in an amount of between about 2% and about 6% by weight of the compounded topical composition wherein the antiviral component comprises acyclovir and the local anesthetic component is lidocaine. The above methods may further include combining additional antiviral component, local anesthetic component, or topical base component to obtain the desired percentage compositions. The additional portions of the components added may be in the form powders, solutions, suspensions, creams, e.g., colloid, emulsion, gel, ointment, lotion, cream, or combinations thereof, for example.

The above methods may further comprise combining an additional active component comprising an anti-inflammatory component with the topical base component or topical base composition to prepare the compounded topical composition. The anti-inflammatory component may include one or more NSAIDS. The anti-inflammatory component may include one or more of the active ingredients added in the form of a solution, such as a diclofenac solution, suspension, emulsion, or fine powder, such as from ground oral tablets or bulk powder. In one example, the compounded topical composition includes between about 1% and about 5% by weight anti-inflammatory component wherein the anti-inflammatory component comprises at least one NSAID selected from meloxicam, piroxicam, flurbiprofen, ketoprofen, naproxen, oxaprozin, diclofenac, nabumetone. Other anti-inflammatory actives or amounts may be used, including those discussed elsewhere herein.

The above methods may comprise combining an additional active component comprising an anticonvulsant component to the topical base component or topical base composition to form the compounded topical composition. The anticonvulsant component may include one or more of the active ingredients added in the form of a solution, suspension, emulsion, or fine powder. In one example, the compounded topical composition includes between about 1% and about 5% by weight anticonvulsant component wherein the anticonvulsant component comprises at least one of lamotrigine or topiramate. Other anticonvulsant actives and amounts may be used, including those discussed elsewhere herein.

The above method may comprise combining an additional active component comprising a nerve depressant component to the topical base component or topical base composition to form the compounded topical composition. The nerve depressant component may include one or more of the active ingredients in the form of a solution, suspension, emulsion, or fine powder. In one example, the compounded topical composition includes between about 1% and about 5% by weight nerve depressant component wherein the nerve depressant component comprises gabapentin. Other nerve depressant actives and amounts may be used, including those discussed elsewhere herein.

The method may comprise combining an additional active component comprising a muscle relaxant component to the topical base component or topical base composition to form the compounded topical composition. The muscle relaxant component may include one or more of the active ingredients added in the form of a solution, suspension, emulsion, or fine powder. In one example, the compounded topical composition includes between about 1% and about 5% by weight muscle relaxant component wherein the muscle relaxant component comprises cyclobenzaprine. Other muscle relaxant actives and amounts may be used, including those discussed elsewhere herein.

The method may comprise combining an additional active component comprising a NMDA receptor antagonist component to the topical base component or topical base composition to form the compounded topical composition. The NMDA receptor antagonist component may include one or more of the active ingredients added in the form of a solution, suspension, emulsion, or fine powder. In one example, the compounded topical composition includes between about 1% and about 5% by NMDA receptor antagonist component wherein the NMDA receptor antagonist component comprises ketamine. Other NMDA receptor antagonist actives and amounts may be used, including those discussed elsewhere herein.

The method may comprise combining an additional active component comprising an opiate or opioid agonist component to the topical base component or topical base composition to form the compounded topical composition. The opiate or opioid agonist component may include one or more of the active ingredients added in the form of a solution, suspension, emulsion, or fine powder. In one example, the compounded topical composition includes between about 1% and about 5% by weight opiate or opioid agonist component wherein the opiate or opioid agonist component comprises one or more of the C2 and C3 opiate agonists named above, tramadol, or a combination thereof. Other opiate or opioid agonist actives and amounts may be used, including those discussed elsewhere herein.

In one embodiment, the method may include addition of excipients to the topical base component or topical base composition to form the compounded topical composition. In another embodiment, the method does not include addition of excipients to the topical base component or topical base composition. For example, in one embodiment the topical base component includes is provided in a commercially manufactured/available composition containing all or a portion of the local anesthetic component, such as Lidocaine and Prilocaine Cream, gel, ointment, lotion, etc. or lidocaine cream, gel, ointment, lotion, etc., and the method does not include addition of excipients, e.g., additional excipients to the topical base component.

A method of compounding active components with a topical base component using a fine powder of one or more actives is disclosed herein. In this embodiment, a topical base component may be a topical base composition, such as a commercially manufactured/available Lidocaine Cream/Ointment or Lidocaine and Prilocaine Cream. The preparer, such as a pharmacist, may calculate the weight of powders needed. Then, the preparer may grind the actives, such as oral tablets containing one or more of the actives, into fine powder and weigh the ingredients. The preparer may bring to total weight with the Lidocaine Cream/Ointment or Lidocaine and Prilocaine Cream and mix well. The mixture may be milled in an ointment mill as necessary to acquire the desired smooth consistency. After which, the preparer may mix thoroughly and package appropriately in sterile tubes.

In a further embodiment, the method may include selecting a topical base component for a transdermal cream, gel, or ointment. The topical base component may include a topical base composition that includes all or a portion of the local anesthetic component, such as one or more local anesthetics, e.g., lidocaine or lidocaine and prilocaine, or all or a portion of the antiviral component, such as one or more antivirals, e.g., Acyclovir 5% Ointment. In one example, the topical base component may include about equal amounts of lidocaine and prilocaine, such as a Lidocaine and Prilocaine 2.5%/2.5% Cream (e.g., EMLA cream or a generic). In another example, the topical base component may include lidocaine, such as a Lidocaine 5% Ointment. Other initial amounts or strengths of lidocaine and/or prilocaine or acyclovir may be used. The method may further include calculating an amount of actives needed and grinding up a suitable amount of the oral tablets containing the actives to obtain the calculated amount of actives for the batch. The method may further include combining the fine powder of ground oral tablets with the topical base component and milling in an ointment mill and mixing.

An example application of the above method with respect to a compounded topical composition comprising lidocaine 2.25% by weight, prilocaine 2.25% by weight, and acyclovir 8% by weight is provided. The antiviral component comprises acyclovir obtained from grinding 800 mg acyclovir oral tablets. The local anesthetic component comprises lidocaine and prilocaine obtained from a commercially manufactured/available Lidocaine and Prilocaine 2.5%/2.5% Cream that includes the topical base component. To calculate the weight of the powders needed, each gram of compounded topical component contains 8% acyclovir (or 80 mg acyclovir) which is equivalent to 0.1005 acyclovir 800 mg oral tablets, which is equivalent to 100.5 mg total weight of acyclovir 800 mg oral tablets. Based on the determination of the amount of tablets required to obtain the desired amount of active, the number of tablets needed may be multiplied by the average weight of a tablet to obtain the weight of the tablet powder needed. The tablets are ground into a fine powder and weighed to obtain the desired weight of powder before combining. The fine powder is combined with the topical base composition comprising the Lidocaine and Prilocaine 2.5%/2.5% Cream. To calculate the weight of the cream needed, each gram of compounded topical composition comprises 2.25% lidocaine and 2.25% prilocaine, which is equivalent to 0.9 g Lidocaine and Prilocaine 2.5%/2.5% Cream. The cream and acyclovir fine powder may be combined in an appropriate container, such as an electronic mixer jar and mixed once on normal setting. The mixture may then be milled. An Exakt 120S-450 three roll mill, front roller “1”, rear roller “3” is suitable. The milled mixture may then be mixed once in the electronic mixer on normal setting. The resulting compounded topical composition may then be packaged in appropriate containers, such as tubes, which may be sterile tubes.

Another example application of the above method with respect to a compounded topical composition comprising lidocaine 3.5% by weight and acyclovir 8% by weight is provided. The antiviral component is acyclovir obtained from grinding 800 mg acyclovir oral tablets. The local anesthetic component is Lidocaine Hydrochloride USP Monohydrate (bulk powder). The topical base component is PCCA PracaSil™-Plus. To calculate the weight of the powders needed, each gram of compounded topical component contains 8% acyclovir (or 80 mg acyclovir) which is equivalent to 0.1005 acyclovir 800 mg oral tablets, which is equivalent to 100.5 mg total weight of acyclovir 800 mg oral tablets. Based on the determination of the amount of tablets required to obtain the desired amount of active, the number of tablets needed may be multiplied by the average weight of a tablet to obtain the weight of the tablet powder needed. The tablets are ground into a fine powder and weighed to obtain the desired weight of powder before combining. Each gram of compounded topical composition comprises 0.043 gm of Lidocaine Hydrochloride USP Monohydrate (1 gm lidocaine base is equivalent to 1.23 gm Lidocaine HCl Monohydrate). Each gram of compounded topical composition comprises 0.8575 gm of PCCA PracaSil™-Plus. The fine powders of acyclovir crushed tablets and Lidocaine HCl Monohydrate are combined with the PCCA PracaSil™-Plus in an appropriate container, such as an electronic mixer jar and mixed once on normal setting. The mixture may then be milled. An Exakt 120S-450 three roll mill, front roller “1”, rear roller “3” is suitable. The milled mixture may then be mixed once in the electronic mixer on normal setting. The resulting compounded topical composition may then be packaged in appropriate containers, such as tubes, which may be sterile tubes.

Another example application of the above method with respect to a compounded topical composition comprising lidocaine 5% by weight and acyclovir 8% by weight is provided. The antiviral component comprises acyclovir obtained from grinding 800 mg acyclovir oral tablets. The local anesthetic component comprises lidocaine obtained from a commercially manufactured/available Lidocaine 5% Ointment that includes the topical base component. To calculate the weight of the powders needed, each gram of compounded topical component contains 8% acyclovir (or 80 mg acyclovir) which is equivalent to 0.1005 acyclovir 800 mg oral tablets, which is equivalent to 100.5 mg total weight of acyclovir 800 mg oral tablets. Based on the determination of the amount of tablets required to obtain the desired amount of active, the number of tablets needed may be multiplied by the average weight of a tablet to obtain the weight of the tablet powder needed. The tablets are ground into a fine powder and weighed to obtain the desired weight of powder before combining. The fine powder is combined with the topical base/Lidocaine 5% Ointment. To calculate the weight of the ointment needed, each gram of compounded topical composition comprises 4.5% lidocaine which is equivalent to 0.9 g Lidocaine 5% Ointment. The ointment and acyclovir fine powder may be combined in an appropriate container, such as an electronic mixer jar and mixed once on normal setting. The mixture may then be milled. An Exakt 120S-450 three roll mill, front roller “1”, rear roller “3” is suitable. The milled mixture may then be mixed once in the electronic mixer on normal setting. The resulting compounded topical composition may then be packaged in appropriate containers, such as tubes, which may be sterile tubes.

Another example application of the above method with respect to a compounded topical composition comprising lidocaine 4.25% by weight and acyclovir 12% by weight is provided. The antiviral component comprises acyclovir obtained from grinding 800 mg acyclovir oral tablets. The local anesthetic component comprises lidocaine obtained from a commercially manufactured/available Lidocaine 5% Ointment that includes the topical base component. To calculate the weight of the powder needed, each gram of compounded topical component contains 12% acyclovir (or 120 mg acyclovir) which is equivalent to 0.1508 acyclovir 800 mg oral tablets, which is equivalent to 150.8 mg total weight of acyclovir 800 mg oral tablets. Based on the determination of the amount of tablets required to obtain the desired amount of active, the number of tablets needed may be multiplied by the average weight of a tablet to obtain the weight of the tablet powder needed. The tablets are ground into a fine powder and weighed to obtain the desired weight of powder before combining. The fine powder is combined with the topical base/Lidocaine 5% Ointment. To calculate the weight of the ointment needed, each gram of compounded topical composition comprises 4.25% lidocaine which is equivalent to 0.85 g Lidocaine 5% Ointment. The ointment and acyclovir fine powder may be combined in an appropriate container, such as an electronic mixer jar and mixed once on normal setting. The mixture may then be milled. An Exakt 120S-450 three roll mill, front roller “1”, rear roller “3” is suitable. The milled mixture may then be mixed once in the electronic mixer on normal setting. The resulting compounded topical composition may then be packaged in appropriate containers, such as tubes, which may be sterile tubes.

Another example application of the above method with respect to a compounded topical composition comprising lidocaine 2.5% by weight and acyclovir 4.8% by weight is provided. The local anesthetic component is Lidocaine Hydrochloride USP Monohydrate (bulk powder). The antiviral component and topical base component are provided together in Acyclovir 5% Ointment. Each gram of compounded topical composition comprises 2.5% or 25 mg lidocaine, which is equivalent to 0.0308 gm of Lidocaine Hydrochloride USP Monohydrate (1 gm lidocaine base is equivalent to 1.23 gm Lidocaine HCl Monohydrate). Each gram of compounded topical composition comprises 96.7% or 0.9666 gm of Acyclovir 5% Ointment. The Lidocaine HCl Monohydrate powder is combined with the Acyclovir 5% Ointment in an appropriate container, such as an electronic mixer jar and mixed once on normal setting. The mixture may then be milled. An Exakt 120S-450 three roll mill, front roller “1”, rear roller “3” is suitable. The milled mixture may then be mixed once in the electronic mixer on normal setting. The resulting compounded topical composition may then be packaged in appropriate containers, such as tubes, which may be sterile tubes.

### III. Methods of Administration and Treatment

As described above, a method of treating various viral ailments related to herpes simplex and varicella zoster. Treatment may include topically administering a sufficient amount of the compounded topical composition described herein to an infected skin or mucosal area or area in which infection is sought to be prevented. The composition may be rubbed into site or may be applied using a dressing or patch. Administration at the site may be repeated twice a day, daily, for a duration of time as needed for preventative treatment or treatment of a current infection. Treatment with the compounded topical composition may reduce frequency and severity of outbreaks. Treatment with the compounded topical composition may also reduce duration of outbreaks in infected individuals. Treatment with the compounded topical composition may reduce risk of acquisition or transmission of the virus. In some embodiments, treatment with the compounded topical composition may reduce shedding.

The grammatical articles “one”, “a”, “an”, and “the”, as used in this specification, are intended to include “at least one” or “one or more”, unless otherwise indicated. Thus, the articles are used in this specification to refer to one or more than one (i.e., to “at least one”) of the grammatical objects of the article. By way of example, “a component” means one or more components, and thus, possibly, more than one component is contemplated and may be employed or used in an application of the described embodiments. Further, the use of a singular noun includes the plural, and the use of a plural noun includes the singular, unless the context of the usage requires otherwise.

This disclosure describes various elements, features, aspects, and advantages of various embodiments, examples, formulations, configurations, and arrangements of the oven systems, apparatuses, and methods thereof. It is to be understood that certain descriptions of the various embodiments and such configurations and arrangements thereof have been simplified to illustrate only those elements, features and aspects that are relevant to a more clear understanding of the disclosed embodiments, while eliminating, for purposes of brevity or clarity, other elements, features and aspects. Any references to “various,” “certain,” “some,” “one,” or “an” when followed by “embodiment,” “example,” “formulation,” “instance,” “configuration,” or “arrangement” generally means that a particular element, feature or aspect described in the example is included in at least one embodiment. The phrases “in various,” “in certain,” “in some,” “in one,” or “in an” when followed by “embodiment”, “configuration”, or “arrangement” may not necessarily refer to the same embodiment. Furthermore, the phrases “in one such” or “in this” when followed by “embodiment,” “configuration,” or “arrangement,” while generally referring to and elaborating upon a preceding embodiment, is not intended to suggest that the elements, features, and aspects of the embodiment introduced by the phrase are limited to the preceding embodiment; rather, the phrase is provided to assist the reader in understanding the various elements, features, and aspects disclosed herein and it is to be understood that those having ordinary skill in the art will recognize that such elements, features, and aspects presented in the introduced embodiment may be applied in combination with other various combinations and sub-combinations of the elements, features, and aspects presented in the disclosed embodiments. It is to be appreciated that persons having ordinary skill in the art, upon considering the descriptions herein, will recognize that various combinations or sub-combinations of the various embodiments and other elements, features, and aspects may be desirable in particular implementations or applications. However, because such other elements, features, and aspects may be readily ascertained by persons having ordinary skill in the art upon considering the description herein, and are not necessary for a complete understanding of the disclosed embodiments, a description of such elements, features, and aspects may not be provided. For example, ovens and oven systems described herein may also include connections such as fittings for one or more of electrical connections, gas connections, or flue connections. As such, it is to be understood that the description set forth herein is merely exemplary and illustrative of the disclosed embodiments and is not intended to limit the scope of the invention as defined solely by the claims.

What is claimed is:

1. A method of compounding a compounded topical composition, the method comprising:
combining a local anesthetic component with a topical base composition, wherein the local anesthetic component comprises lidocaine hydrochloride monohydrate and the topical base composition comprises an Acyclovir 5% Ointment containing 5% acyclovir by weight and polyethylene glycol.

2. The method of claim 1, wherein the local anesthetic component is combined with the topical base composition in an amount such that the compounded topical composition comprises between about 2% and about 4.75% lidocaine by weight and between about 2% and about 4.9% acyclovir by weight.

3. The method of claim 1, wherein the local anesthetic component is combined with the topical base composition in an amount such that the compounded topical composition comprises about 2.5% lidocaine by weight and about 4.8% acyclovir by weight.

4. The method of claim 1, further comprising combining an anti-inflammatory component comprising at least one NSAID active selected from meloxicam, piroxicam, flurbiprofen, ketoprofen, naproxen, oxaprozin, diclofenac, nabumetone, or combination thereof, wherein the anti-inflammatory component is combined in an amount between about 1% and about 5% by weight of the compounded topical composition.

5. The method of claim 4, further comprising grinding one or more oral tablets of the NSAID active to obtain a fine powder, and wherein combining the anti-inflammatory component comprises combining the fine powder of the one or more oral tablets of the NSAID active with the topical base composition.

6. The method of claim 4, further comprising combining at least one additional active component selected from (i) an anticonvulsant component comprising at least one anticonvulsant active, (ii) a nerve depressant component comprising at least one depressant active, (iii) a muscle relaxant component comprising at least one muscle relaxant active, (iv) a NMDA receptor antagonist component comprising at least one NMDA receptor antagonist active, (v) an opiate or opioid agonist component comprising at least one opiate or opioid agonist active, or (vi) any combination thereof, wherein the additional active component is combined in an amount between about 1% and about 5% by weight of the compounded topical composition.

7. The method of claim 6, further comprising grinding one or more oral tablets of the active of the additional active component to obtain a fine powder of the oral tablets comprising the active of the additional active component, and wherein combining the additional active component comprises combining the fine powder of the oral tablets comprising the active of the additional active component with the topical base composition.

8. A method of compounding a compounded topical composition, the method comprising:
grinding one or more oral tablets of acyclovir to obtain a fine powder; and
combining the fine powder with a local anesthetic component and a topical base component, wherein the topical base component comprises an anhydrous silicone gel comprising pracaxi oil and over 50% silicones, and wherein the local anesthetic component comprises lidocaine hydrochloride monohydrate.

9. The method of claim 8, wherein the fine powder and the local anesthetic components are combined with the topical base component in amounts such that the compounded topical composition comprises between about 2% and about 5% lidocaine by weight and between about 4% and about 14% acyclovir by weight.

10. The method of claim 8, wherein the fine powder and the local anesthetic component are combined with the topical base component in amounts such that the compounded topical composition comprises about 3.5% lidocaine by weight and about 8% acyclovir by weight.

11. The method of claim 8, further comprising combining an anti-inflammatory component comprising at least one NSAID active selected from meloxicam, piroxicam, flurbiprofen, ketoprofen, naproxen, oxaprozin, diclofenac, nabumetone, or combination thereof, wherein the anti-inflammatory component is combined in an amount between about 1% and about 5% by weight of the compounded topical composition.

12. The method of claim 11, further comprising grinding one or more oral tablets of the NSAID active to obtain a fine powder, and wherein combining the anti-inflammatory component comprises combining the fine powder of the one or more oral tablets of the NSAID active with the topical base composition.

13. The method of claim 11, further comprising combining at least one additional active component selected from an anticonvulsant component comprising at least one anticonvulsant active, a nerve depressant component comprising at least one depressant active, a muscle relaxant component comprising at least one muscle relaxant active, a NMDA receptor antagonist component comprising at least one NMDA receptor antagonist active, an opiate or opioid agonist component comprising at least one opiate or opioid agonist active, or any combination thereof, wherein the additional active component is combined in an amount between about 1% and about 5% by weight of the compounded topical composition.

14. The method of claim 13, further comprising grinding one or more oral tablets of the active of the additional active component to obtain a fine powder of the oral tablets comprising the active of the additional active component, and wherein combining the additional active component comprises combining the fine powder of the oral tablets comprising the active of the additional active component with the topical base component.

15. A method of producing a compounded topical composition, the method comprising:
grinding one or more oral tablets comprising the antiviral acyclovir to obtain a fine powder of the one or more oral tablets; and
combining the fine powder with a topical base composition comprising Lidocaine 5% Ointment.

16. The method of claim 15, wherein the fine powder is combined with the topical base composition in an amount such that the compounded topical composition comprises between about 2% and about 4.75% lidocaine by weight and between about 4% and about 14% acyclovir by weight.

17. The method of claim 15, wherein the fine powder is combined with the topical base composition in an amount such that the compounded topical composition comprises about 4.25% lidocaine by weight and about 14% acyclovir by weight.

18. The method of claim 15, further comprising combining an anti-inflammatory component comprising at least one NSAID active selected from meloxicam, piroxicam, flurbiprofen, ketoprofen, naproxen, oxaprozin, diclofenac, nabumetone, or combination thereof, wherein the anti-inflammatory component is combined in an amount between about 1% and about 5% by weight of the compounded topical composition.

19. The method of claim 18, further comprising grinding one or more oral tablets of the NSAID active to obtain a fine powder, and wherein combining the anti-inflammatory component comprises combining the fine powder of the one or more oral tablets of the NSAID active with the topical base composition.

20. The method of claim 18, further comprising combining at least one additional active component selected from an anticonvulsant component comprising at least one anticonvulsant active, a nerve depressant component comprising at least one depressant active, a muscle relaxant component comprising at least one muscle relaxant active, a NMDA receptor antagonist component comprising at least one NMDA receptor antagonist active, an opiate or opioid agonist component comprising at least one opiate or opioid agonist active, or any combination thereof, wherein the additional active component is combined in an amount between about 1% and about 5% by weight of the compounded topical composition.
